(19) United States
(12) Patent Application Publication
(10) Pub. No.: US 2021/0369631 A1
(43) Pub. Date: Dec. 2, 2021

(54) A LIPID-POLYMER HYBRID NANOPARTICLE

(71) Applicant: NANOBRID INNOVATIONS PRIVATE LIMITED, Punjab (IN)

(72) Inventors: Deepak Chitkara, Rajasthan (IN); Sudeep Sudesh Pukale, Rajasthan (IN); Arihant Kumar Singh, Rajasthan (IN); Anupama Mittal, Rajasthan (IN); Saurabh Sharma, Rajasthan (IN)

(21) Appl. No.: 17/284,155

(22) PCT Filed: Feb. 2, 2020
(86) PCT No.: PCT/IB2020/050819

§ 371 Date: Apr. 9, 2021

(30) Foreign Application Priority Data
Feb. 2, 2019 (IN) 201921004214

(51) Int. Cl. A61K 9/51; A61K 45/06

(52) U.S. Cl. CPC A61K 9/5123; B82Y 5/00; A61K 9/513; A61K 45/06

(57) ABSTRACT

The present invention provides a lipid-polymer hybrid nanoparticles of a hydrophobic drug molecules. Particularly the present invention provides a lipid-polymer hybrid nanoparticle comprising a solid lipid, a liquid lipid and an amphiphilic polymer.

### FIELD OF THE INVENTION

The present invention relates to a lipid-polymer hybrid nanoparticles of hydrophobic drug molecules. Particularly the present invention provides a lipid-polymer hybrid nanoparticle comprising a solid lipid, a liquid lipid and an amphiphilic polymer.

### BACKGROUND

Most of the drug molecules that have been discovered and are being used in pharmaceuticals, nutraceuticals and cosmetics are hydrophobic in nature thereby posing challenges in their delivery. Further they exhibit lower efficacy and high toxicity due to their non-specific distribution and higher dose requirement. To overcome this problem, drug delivery using nano-carriers are being approached that includes polymeric (polymeric nanoparticles, polymeric micelles and polymer-drug conjugate, etc) and non-polymeric i.e. lipidic nano-carriers. These nano-systems offer many benefits over conventional delivery systems including prolonged drug release profile, protection of the active principle from the destructive bio-environment, lower dose leading to lesser side effects, targeting drug to the active site and altering the pharmacokinetic parameters. Both polymeric and non-polymeric nanoparticles have been extensively reported to offer several advantages however certain disadvantages are also associated with these systems. Polymeric nanoparticles offer advantages such as lower particle size with narrow size distribution, possible chemical modifications over the surface, prolonged drug release and stability. But at the same time these are associated with disadvantages such as lower drug entrapment, multiple steps that are involved in the preparation method, use of large quantities of organic solvents, scalability and cost of manufacturing. Non-polymeric nano-systems include nanosuspensions, nanocrystals, microemulsions, solid lipid nanoparticles (SLN), nanostructure lipid carriers (NLC), liposomes, self-emulsifying drug delivery systems (SEDDS), niosomes, etc. These types of systems offer advantages such as lower cost of manufacturing, higher encapsulation efficiencies, less number of steps that are involved and no toxicity issues. These are too associated with the limitations including burst release, limited opportunities for chemical modifications, instability and high polydispersity index, drug partitioning, drug expulsion, etc.

In order to get benefits of both systems, lipid-polymer hybrid nano-systems have been developed. These newer class of nano-carriers combines advantages of both polymeric and lipidic nano-carriers such as good drug loading capacities, a more controlled drug release, improved cellular uptake and biocompatibility avoiding the disadvantages associated with them.

These characteristics of lipid-polymer hybrid (LPH) nanoparticles have encouraged their applications in the delivery of chemotherapeutics, proteins, peptides and vaccines.

US20130315831A1 discloses a PLGA-DSPE-PEG based hybrid particulate carrier system comprising of an aqueous core surrounded by an amphiphilic layer of lipid which was further covered by a polymeric matrix. These particles are proposed for encapsulation and delivery of siRNA, anti-cancer drugs like doxorubicin and imaging agents for theranostics. The said hybrid particulate was prepared by emulsion solvent evaporation by using dichloromethane (DCM) as an organic solvent.

US20080102127A1 discloses a hybrid nanoparticles prepared by using polymers (PLGA/PCL) and lipids (like tristearin, tripalmitin, glycerin stearate, cholesterol, tocopherol palmitate etc.) and high quantities of surfactants (Tween 80, cremophor EL, TPGS, pluronics, PVA). In this invention, hybrid nano-systems are made by nanoprecipitation and emulsion solvent evaporation methods. US20100203142A1 discloses the use of lipid-polymer hybrid nanoparticles comprising of pegylated or non-pegylated polymers and amphiphilic lipid. Here the amphiphilic lipid coats the outer surface of nanoparticle forming shell and polymer forms the core. The lipids used are made up of both hydrophilic and hydrophobic moieties (phospholipids). Here the hybrid nanoparticles prepared by method similar to that of emulsion solvent evaporation.

CN107412191A discloses the use of polymers and cationic lipids that form shell enclosing the central aqueous core that contains drug. WO2017158093A1 discloses the use of lipidoids (lipids containing secondary and tertiary amines) and PLGA polymers for making hybrid systems.

WO2013033513A1 discloses the use of hybrid systems made up of polymers (that form hydrophobic core and with outer hydrophilic portion) and phospholipids that assembles at the outer surface of core forming coat. Here inventors had used nanoprecipitation method for the preparation of hybrid nanoparticles.

US20140005269A1 discloses polymer-lipid nanoparticles incorporated within a polymeric matrix for delivery of a poorly soluble drug Levodopa to treat Parkinson's disease. Here the polymer-lipid nanoparticles comprises of at least one polymer, such as Eudragit® E100 and/or chitosan, and at least one phospholipid, such as lecithin. It also includes, the polymer matrix formed from at least two crosslinked cationic and anionic polymers, such as Eudragit® E100 and sodium carboxymethylcellulose.

There is still unmet need to provide novel lipid-polymer hybrid nanoparticles having high efficient entrapment of hydrophobic active molecules with better stability, prolonged drug release with minimal burst release, simple and scalable process prepared using biocompatible excipients having low/minimal toxicity and prevention of drug leakage, drug partitioning and drug expulsion effect.

### SUMMARY OF THE INVENTION

The present invention provides a hybrid system comprises of a polymer, a solid lipid, a liquid lipid and a surfactant. The said lipid-polymer hybrid nanoparticles can serve as a platform for delivering various hydrophobic molecules. The most important advantage of the proposed systems is that it can serve as a platform technology, wherein the different hydrophobic drug molecules can be loaded in a matrix that is made up of the selected class of excipients (solid and liquid lipid, polymer and surfactant).

The present invention also discloses the method of preparation of lipid-polymer hybrid nanoparticles that are scalable to commercial level and uses processes which could be easily adapted for industrial application.

The lipid-polymer hybrid nanoparticles, according to the present invention comprises of a hydrophobic lipids (solid lipid and liquid lipid) and an amphiphilic polymeric matrix, wherein the hydrophobic block of amphiphilic copolymer and the lipids form the core, while the hydrophilic block of the polymer forms a hydrophilic shell around the nanoparticles forming monolithic lipid-polymer hybrid (LPH) nanoparticles. Further the particles are stabilized using a biocompatible surfactant. For the purpose of the present invention a hydrophobic drug is entrapped in the core of the particles. Due to said specific structural arrangement of components of LPH nanoparticles of the present invention it results into better drug loading and encapsulation efficiency with prolonged drug release profile. Since the core of the particle is made up of spatially different components, it will be devoid of drug expulsion effect as seen in solid-lipid nanoparticles. Further, due to the presence of hydrophilic surface these particles could show better efficacy owing to excellent skin retention property when used topically and could show enhanced circulation time of drug in body by the stealth effect when administered by parenteral route.

### DESCRIPTION OF DRAWINGS

Many aspects of this disclosure can be better understood with reference to the following drawings. The components in the drawings are not necessarily to scale, emphasis instead being placed upon clearly illustrating the principles of the present disclosure.

FIG. 1. Particle size distribution of CP loaded LPH nanoparticles (Batch 5) prepared using probe sonicator.

FIG. 2. CP loaded LPH nanoparticles (Batch 5) A) Undiluted and B) 100× diluted and C) particle size distribution of clobetasol propionate loaded LPH nanoparticles prepared by high pressure homogenizer.

FIG. 3. In-vitro drug release studies of free clobetasol propionate (CP), gel containing free clobetasol propionate (CP), gel containing clobetasol propionate loaded LPH nanoparticles (CP LPH NP) of batch 1 and 5 prepared using high pressure homogenizer.

FIG. 4. In-vitro drug release studies of free clobetasol propionate (CP), gel containing free clobetasol propionate (CP), gel containing clobetasol propionate loaded LPH nanoparticles (CP LPH NP), gel containing clobetasol propionate loaded LPH nanoparticles (CP LPH NP) stored at 2-8° C. and room temperature for 3 months and 6 months.

FIG. 5. In-vitro drug release studies of free docetaxel (DTX) and DTX loaded LPH nanoparticle (DTX LPH NP) prepared using probe sonicator.

FIG. 6. In-vitro cytotoxicity of A) free clobetasol propionate (CP) and clobetasol propionate loaded LPH nanoparticles (CP LPH NP), B) free cholecalciferol (CVD) and cholecalciferol loaded LPH nanoparticles (CVD LPH NP) in HaCaT cells and C) free docetaxel (DTX) and docetaxel loaded LPH nanoparticle (DTX LPH NP) in 4T1 breast cancer cells.

FIG. 7. Cumulative score indicating extent of psoriatic inflammation based on the clinical Psoriasis Area and Severity Index (PASI) developed in animals treated with A) Clobetasol propionate (CP) gels, B) Cholecalciferol (CVD) gels and C) Coenzyme Q10 (CoQ10) gels in imiquimod induced psoriasis model in swiss albino mice.

FIG. 8. Histopathological (H&E staining) evaluation of Ear skin (ES) and Back skin of animals treated with clobetasol propionate (CP) containing gels. Magnification 40×. RED Arrow: Epidermis; Plain green arrows: Dermis; Black arrow: Stratum Corneum; Yellow Arrow: Infiltration of inflammatory Cells; Blue Arrow: Capillary Proliferation; Orange Arrow: Epidermal Hyperplasia.

FIG. 9. Histopathological (H&E staining) evaluation of Ear skin (ES) and Back skin of animals treated with Cholecalciferol gels (CVD) containing gels. Magnification 40×. RED Arrow: Epidermis; Plain green arrows: Dermis; Black arrow: Stratum Corneum; Yellow Arrow: Infiltration of inflammatory Cells; Blue Arrow: Capillary Proliferation; Orange Arrow: Epidermal Hyperplasia; White Arrow: Parakeratosis; Fluorescent green arrow: Hyperkeratosis; Sky blue arrow: Munro microabscess; Pink arrow: Pustule of Kogoj.

FIG. 10. Histopathological (H&E staining) evaluation of Ear skin (ES) and Back skin of animals treated with Coenzyme Q10 (CoQ10) containing gels. Magnification 40×. RED Arrow: Epidermis; Plain green arrows: Dermis; Black arrow: Stratum Corneum; Yellow Arrow: Infiltration of inflammatory Cells; Blue Arrow: Capillary Proliferation; Orange Arrow: Epidermal Hyperplasia; White Arrow: Parakeratosis; Fluorescent green arrow: Hyperkeratosis.

FIG. 11. Quantification of clobetasol propionate (CP) in remaining skin (RS) i.e. viable epidermis and dermis after topical application of clobetasol propionate formulations.

### DETAILED DESCRIPTION

It was surprisingly found that the use of a combination of solid lipid, liquid lipid and an amphiphilic polymer resulted in specific structured LPH nanoparticles having a hydrophobic core made up of lipids and hydrophobic segment of polymer which was covered by a hydrophilic shell consisting of hydrophilic segment of polymer. The said arrangement of components resulted in stable LPH nanoparticles showing high drug loading capacities, improved entrapment efficiencies, prolonged drug release and no burst effect. Further manufacturing process can be easily scaled up using high pressure homogenizer.

As used herein, “nanoparticle” is generally referred to both nano-scale and micro-scale particles and, except where otherwise noted, is generally synonymous with the term “particle”.

It is also specifically understood that any numerical value recited herein includes all values from the lower value to the upper value, i.e., all possible combinations of numerical values between the lowest value and the highest value enumerated are to be considered to be expressly stated in this application. For example, if a concentration range or a beneficial effect range is stated as 1% to 50%, it is intended that values such as 2% to 40%, 10% to 30%, or 1% to 3%, etc., are expressly enumerated in this specification. These are only examples of what is specifically intended.

The lipid-polymer hybrid nanoparticles (LPH) according to the present invention comprises a hydrophobic core made up of solid lipid, liquid lipid and hydrophobic segment of an amphiphilic polymer which is surrounded by a hydrophilic portion of an amphiphilic polymer.

The lipid-polymer hybrid nanoparticles (LPH) according to the present invention comprises a solid lipid, a liquid lipid and an amphiphilic polymer.

In one embodiment, the present invention describes LPH wherein a solid lipid, a liquid lipid and an amphiphilic polymer form monolithic type hybrid nanoparticles wherein hydrophobic block of polymer interacts with both a solid lipid and a liquid lipid to form a hydrophobic core and the hydrophilic portion of polymer arranges itself outside the hydrophobic core.

In another embodiment, LPH nanoparticles according to the present invention comprises a solid lipid, a liquid lipid and an amphiphilic polymer, wherein (a) core comprises of a solid lipid, a liquid lipid and hydrophobic block of polymer and (b) shell surrounding the core comprises of a hydrophilic portion of polymer.

As per another aspect, LPH nanoparticles according to the present invention comprises a solid lipid, a liquid lipid, an amphiphilic polymer and a surfactant.

As per another aspect, LPH nanoparticles according to the present invention comprises at least one solid lipid, at least one liquid lipid and at least one amphiphilic polymer and at least one surfactant.

As per another embodiment, LPH nanoparticles having z-average diameter between 10-500 nm. Owing to nano-metric size, the stated systems can be administered into systemic circulation without particle aggregation or blockage, exhibit higher intracellular uptake, can penetrate submucosal layers, can exhibit increased oral bioavailability, can demonstrate selective targeting, improved efficacy and decreased dose requirement. On topical application, they shown deeper skin penetration thus exhibit improved efficacy of active principle and are retained in the skin layers for longer duration with minimal systemic absorption.

For the purpose of the present invention at least one solid lipid is selected from but not limited to glycerides (such as glyceryl palmitostearate (Precirol®), glyceryl monostearate (GMS), glyceryl behenate (Comptritol ATO 888,®), Gelucire® etc, Wax (Apifil®), Fatty acids (stearic acid, palmitic acid etc.), Sterol (cholesterol and its derivatives etc.), bile acid and its salts (cholic acid, deoxycholic acid etc. and their suitable derivatives) or combination thereof.

For the purpose of the present invention a solid lipid is present in range from about 1% to about 50%, preferably about 15% to about 45%, more preferably about 20% to about 40%, most preferably about 25% to about 35% by weight of the nanoparticles.

For the purpose of the present invention at least one liquid lipid is selected from but not limited to ester and glycerides form or free form of unsaturated fatty acid, saturated fatty acid, saturated and unsaturated fatty amines and alcohols, or combination thereof.

For the purpose of the present invention, unsaturated fatty acid is selected from but not limited to linoleic acid and oleic acid. For the purpose of the present invention, saturated fatty acid is selected from but not limited to capric acid, caprylic and caproic acid. For the purpose of the present invention, saturated and unsaturated fatty amine and alcohols is selected from but not limited to oleylamine, linoleylamine and cetyl alcohol. Esters and glycerides of saturated and unsaturated fatty acid includes Capmul®, Miglyol®, Captex®, Peceol™, Maisine® CC or combination thereof.

For the purpose of the present invention a liquid lipid is present in range from about 1% to about 50%, preferably about 15% to about 45%, more preferably about 20% to about 40%, most preferably about 25% to about 35% by weight of the nanoparticles.

For the purpose of the present invention a ratio of a solid lipid to a liquid lipid is in range of about 10:1 to about 1:10, about 9:1 to about 1:9, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1 or about 1:1.

For the purpose of the present invention, an amphiphilic polymer is selected from but not limited to any natural or synthetic, biodegradable or non-biodegradable, polymer with both hydrophilic and hydrophobic moieties. In some embodiments, the polymeric matrix includes but not limited to diblock, triblock, multiblock or graft polymers composed of hydrophilic polymers including but not limited to poly(ethylene glycol) (PEG), poly(acrylic acid), polymethyloxazoline, polyisoprene, poly(4-vinyl pyridine), poly(4-vinylpyridinum methyl iodide) and different hydrophobic cores like but not limited to poly(aspartic acid) (PAA), polyesters like poly(lactide-co-glycolic acid) (PLGA), poly(caprolactone) (PCL) and poly(lactic acid) (PLA), polystyrene, poly(2-cinnamoylethyl methacrylate), polydimethylsiloxane, poly(propylene oxide), poly(ethyl ethylene), poly(propylene sulphate), poly(N-isopropylacrylamide), poly(fumaric/sebacic acids), polyphosphazenes or combination thereof. As per the preferred embodiment, an amphiphilic polymer is mPEG-PLA, wherein mPEG molecular weight is in a range from about 500 to about 20000 Da and PLA molecular weight is in range from about 2000-about 20000 Da.

For the purpose of the present invention an amphiphilic polymer is present in amount range from about 1% to about 50%, preferably about 15% to about 45%, more preferably about 20% to about 40%, most preferably about 25% to about 35% by weight of the nanoparticles.

For the purpose of the present invention a ratio of a solid lipid to an amphiphilic polymer is in range of about 10:1 to about 1:10, about 9:1 to about 1:9, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1 or about 1:1.

For the purpose of the present invention a ratio of a liquid lipid to an amphiphilic polymer is in range of about 10:1 to about 1:10, about 9:1 to about 1:9, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1 or about 1:1.

As per another aspect, LPH nanoparticles according to present invention entrap one or more active agents within a hydrophobic core formed due to interaction of a solid lipid, a liquid lipid and hydrophobic block of an amphiphilic polymer.

For the purpose of the present invention at least one surfactant is selected from HLB range <5 to >15 but not limited to span 80, tween 80 and solutol HS 15 or combination thereof. These help in stabilization of lipid-polymer hybrid nanoparticles by adsorption onto its surface and preventing the aggregation of particles and hence enhancing the colloidal stability.

For the purpose of the present invention at least one surfactant is present in amount range from about 0.5% w/v to about 10% w/v.

For the purposes of the present invention, nanoparticles encapsulate one or more pharmaceutically active compound such as endogenous molecule or their analogue, anti-oxidant, antibiotic, anti-neoplastic agent, steroidal hormone, sex hormone, peptide, non-steroidal anti-inflammatory drug (NSAID), antifungal drug, antiviral drug, neuraminidase inhibitor, opioid agonist or antagonist, calcium channel blocker, antiangiogenic drug, diagnostic compound and vaccine or biological.

Various pharmaceutically active drugs can be loaded in this proposed lipid polymer hybrid nanoparticles like endogenous molecule or their analogue, anti-oxidant, antibiotic, anti-neoplastic agent, steroidal hormone, sex hormone, peptide, non-steroidal anti-inflammatory drug (NSAID), antifungal drug, antiviral drug, neuraminidase inhibitor, opioid agonist or antagonist, calcium channel blocker, antiangiogenic drug, diagnostic compound and vaccine or biological.

Steroidal hormone can be selected from the group of corticosteroidal hormones (Hydrocortisone, Progesterone, Prednisolone, Betamethasone, Dexamethasone, fluorinated corticosteroids), anabolic steroids (Retabolil, Nerobolil, Androstenolone, Androstenone, Nandrolol), physiologically equivalent hormones (example Vitamin D) derivatives or combinations thereof.

Anti-neoplastic agent can be selected from anticancer antibiotics (Mitomycin, Daunorubicin, Bleomycin, Dactinomycin, Mitoxantrone, Epirubicin, Doxorubicin, Idarubicin, Valrubicin), Topoisomerase inhibitors (Irinotecan, Topotecan), plant alkaloids and their derivatives (Docetaxel, Etoposide, Paclitaxel, Vinblastine, Vincristine, Vinorelbine, Camptothecin, Vindesine), aromatase inhibitors (Letrozole, Anastrozole), antimetabolites (Gemcitabine, Pemetrexed, Methotrexate, Cladribine, Clofarabine, Raltitrexed, Fludarabine, Fluorouracil, Tioguanine, Capecitabine, Mercaptopurine, Cytarabine).

Various additional drugs can be loaded from different categories such as anti-histaminic (cetirizine, loratadine, astemizole, terfenadine etc), leukotriene receptor antagonist (zafirlukast and montelukast), 5-LOX inhibitor (zileuton), non-steroidal anti-inflammatory drugs (ibuprofen, indomethacin, ketoprofen etc), antioxidant (coenzyme Q10, astaxanthin, lycopene, quercetin, 5-alpha lipoic acid etc), calcineurin inhibitors (tacrolimus or pimecrolimus), de-pigmenting agents (monobenzone, mequinol or hydroquinone), alpha agonists (brimonidine, oxymetazoline or xylometazoline), antibiotics (doxorubicin, levofloxacin, rifampicin, azithromycin), anti-fungal agents (ketoconazole and diflucan), anti-parasitics (permethrin, lindane, malathion or benzyl benzoate, sulfiram, carbaril, crotamiton and phenothrin, Albendazole, Tiabendazole), anti-virals (podophyllum resin and podophyllotoxin, acyclovir etc.), drugs for skin cancer (cisplatin, doxorubicin, 5-fluorouracil (5-FU), capecitabine, topotecan, and etoposide, dacarbazine, paclitaxel, Vinblastine).

As per another embodiment of the present invention, nanoparticles encapsulate one or more pharmaceutically active compound having a log P of in ranges from 1 to 10. For example Vorinostat (log P: 1.44), 5-alpha lipoic acid (log P: 2.1), docetaxel trihydrate (log P: 2.92), clobetasol propionate (log P: 3.49), cholecalciferol (log P: 7.5), coenzyme Q10 (log P: 10).

In some embodiments, the particle surface could be modified by attaching a targeting ligand including but not limited to small molecules (including but not limited folic acid, galactose), peptides (including but not limited RGD, TAT) or proteins (including but not limited transferrin) or imaging agents to the hydrophilic segment of an amphiphilic polymer.

The present invention provides a LPH nanoparticles drug delivery vehicle wherein nanoparticle comprises a hydrophobic lipid and an amphiphilic polymeric matrix.

In one embodiment, a LPH nanoparticles drug delivery system comprises (a) a hydrophobic lipid comprising a solid lipid and a liquid lipid and (b) an amphiphilic polymeric matrix.

As per another embodiment, a LPH nanoparticles according to present invention having drug encapsulation efficiency at least about 50% or more. As per preferred embodiment, a LPH nanoparticle according to the present invention having drug encapsulation efficiency is in range of about 50% to about 100%.

As per preferred embodiment, a LPH nanoparticles according to present invention having drug encapsulation efficiency is about 50%, about 55%, about 60%, about 65%, about 70%, about 75%, about 80%, about 85%, about 90%, about 95% or 100%.

As per another embodiment a LPH nanoparticles drug delivery system comprises:
(a) a hydrophobic core comprising a solid lipid and a liquid lipid; and
(b) an amphiphilic polymer, wherein hydrophobic portion forms a hydrophobic core with a solid lipid, a liquid lipid and hydrophilic portion arranges outside the central hydrophobic core forming hydrophilic shells.

The present invention provides a LPH nanoparticle drug delivery vehicle wherein nanoparticle comprises a solid lipid, a liquid lipid and an amphiphilic polymer wherein (a) a core comprises of a solid lipid, a liquid lipid and hydrophobic block of polymer and (b) shell surrounding the core comprises of hydrophilic portion of polymer.

As per another embodiment, a LPH nanoparticles drug delivery vehicle comprises:
(a) a core comprises of a solid lipid, a liquid lipid and hydrophobic block of polymer;
(b) an active molecule entrapped in core; and
(c) shell surrounding core comprises of hydrophilic portion of polymer.

As per another embodiment, a LPH nanoparticles drug delivery vehicle comprises:
(a) a core comprises of a solid lipid, a liquid lipid and hydrophobic block of polymer;
(b) an active molecule entrapped in core;
(c) shell surrounding core comprises of hydrophilic portion of polymer; and
(d) a surfactant.

As per another embodiment, a LPH nanoparticles drug delivery vehicle comprises:
(a) a core comprises of a solid lipid, a liquid lipid and hydrophobic block of polymer;
(b) an active molecule entrapped in core;
(c) shell surrounding core comprises of hydrophilic portion of polymer; and
(d) a surfactant present in the aqueous phase surrounding the nanoparticles.

As per another embodiment, a LPH nanoparticles drug delivery vehicle consisting essentially of:
(a) a core comprises of a solid lipid, a liquid lipid and hydrophobic block of polymer;
(b) an active molecule entrapped in core;
(c) shell surrounding core comprises of hydrophilic portion of polymer; and
(d) a surfactant present in the aqueous phase surrounding the nanoparticles.

As per another embodiment of the invention, LPH nanoparticles is prepared by a dissolving drug or a pharmaceutically active ingredient along with solid lipid, liquid lipid and amphiphilic polymer in minimum amount of chloroform by slight warming followed by heating to remove chloroform resulting in formation homogeneous matrix. The formed matrix is heated or without heating the aqueous surfactant solution is added and subjected to mixing. This aqueous lipidic dispersion which was kept at temperature range from 1° C. to 70° C. and subjected to size reduction technique (sonication or homogenization) followed by immediate cooling of hot nanoparticle dispersion to get LPH nanoparticles.

As per another embodiment of the invention, a matrix containing drug or a pharmaceutically active ingredient along with solid lipid, liquid lipid and amphiphilic polymer is processed at room temperature and an aqueous surfactant solution is transferred at room temperature to vial containing solid matrix and subjected to probe sonication to obtain nanoparticles. Surfactants used were with HLB range <5 to >15 like span 80, tween 80 and solutol HS 15.

In another aspect, the invention features methods of treating a disorder by administering to a subject a particle (or a composition that includes a plurality of particles) as described herein that includes one or more active agents, wherein the one or more active agents are effective to treat the disorder. The invention also features the use of a particle as described herein that includes one or more active agents in the treatment of a disorder, wherein the one or more active agents are effective to treat the disorder. Further, the said particles could be incorporated in a gel, cream or an ointment carrier for application. Further, the said particles could be adminstered to patient by oral, parenteral, nasal, topical, transdermal or opthalmic route.

The invention is further exemplified and disclosed by the following non-limiting examples:

### Example 1: Clobetasol Propionate Loaded LPH Nanoparticles (CP LPH NP) by Probe Sonicator Method

Batches were prepared by as per Table 1, by combining clobetasol propionate with various solid lipids (stearic acid, glyceryl monostearate, compritol, precirol, cholesterol or cholic acid), liquid lipid (oleic acid, linoleic acid, miglyol, capmul MCM C8 or captex 355), amphiphilic polymer (mPEG-PLA) and surfactants (span 80, tween 80 or solutol HS 15). Batches were carried out at 2.17% Theoretical Drug Loading (TDL) by taking drug (4 mg), solid lipid (60 mg), liquid lipid (60 mg), polymer (60 mg) and surfactant (1.5% w/v).

Clobetasol propionate (4 mg), solid lipid (Precirol® ATO 5; 60 mg), liquid lipid (linoleic acid; 60 mg) and polymer (methoxypolyethyleneglycol-co-polylactic acid co-polymer (mPEG-PLA); 60 mg) were taken in a 5 ml glass vial. To this chloroform (0.4 ml) was added and warmed to 40° C. for obtaining a clear solution. Chloroform was then removed by heating at 70° C. for 30 min to form a uniform matrix. An aqueous solution containing tween 80 (1.5% w/v; 3 ml) was added to the matrix and sonicated at 25% amplitude for 4 min using probe sonicator. The nanodispersion was centrifuged at 5000 rpm for 5 min to remove the unentrapped CP and large particles. The supernatant containing nanoparticles was collected analyzed for particle size and size distribution, and zeta potential using Malvern Zetasizer and drug content in the dispersion using HPLC. Results obtained are shown in table 2. FIG. 1 shows the particle size distribution of CP loaded LPH nanoparticles.

TABLE 1
Formulation batches of clobetasol propionate with various solid lipid, liquid lipid, polymer and surfactants.

| | | | | | | | Zeta | % |
|---|---|---|---|---|---|---|---|---|
| Batch | | | | | Particle size | | potential | Encapsulation |
| No | Solid Lipid | Liquid Lipid | Polymer | Surfactant | (nm) | PDI | (-mV) | efficiency |
| 1 | Glyceryl | Oleic acid | mPEG-PLA | Tween 80 | 186.6 ± 5.04 | 0.345 ± 0.03 | 3.12 ± 0.15 | 82.10 ± 3.66 |
| | monostearate | | | | | | | |
| 2 | Stearic acid | Oleic acid | mPEG-PLA | Tween 80 | 267.1 ± 13.12 | 0.426 ± 0.01 | 4.14 ± 0.62 | 60.12 ± 8.27 |
| 3 | Precirol | Oleic acid | mPEG-PLA | Tween 80 | 144.8 ± 5.02 | 0.331 ± 0.06 | 3.56 ± 0.67 | 85.57 ± 2.31 |
| 4 | Cholesterol | Linoleic acid | mPEG-PLA | Tween 80 | 198.5 ± 7.68 | 0.365 ± 0.01 | 1.44 ± 0.13 | 66.57 ± 0.48 |
| 5 | Precirol | Linoleic acid | mPEG-PLA | Tween 80 | 128.3 ± 1.41 | 0.246 ± 0.01 | 6.13 ± 0.50 | 92.40 ± 3.24 |
| 6 | Cholic acid | Linoleic acid | mPEG-PLA | Tween 80 | 129.7 ± 13.75 | 0.235 ± 0.08 | 6.98 ± 0.33 | 65.71 ± 2.08 |
| 7 | Cholic acid | Capmul MCM | mPEG-PLA | Span 80 | 180.7 ± 5.08 | 0.171 ± 0.02 | 1.26 ± 1.06 | 67.35 ± 1.12 |
| | | C8 | | | | | | |
| 8 | Cholic acid | Miglyol ® | mPEG-PLA | Solutol HS | 119.6 ± 4.97 | 0.386 ± 0.02 | 1.72 ± 0.18 | 61.15 ± 0.73 |
| | | | | 15 | | | | |
| 9 | Cholic acid | Captex 355 | mPEG-PLA | Solutol HS | 111.1 ± 3.72 | 0.252 ± 0.01 | 2.11 ± 0.22 | 70.62 ± 1.49 |
| | | | | 15 | | | | |
| 10 | Precirol | Captex 355 | mPEG-PLA | Solutol HS | 154.5 ± 2.11 | 0.408 ± 0.02 | 0.64 ± 0.27 | 66.47 ± 0.64 |
| | | | | 15 | | | | |
| 11 | — | Oleic acid | — | Tween 80 | 107.0 ± 16.82 | 0.238 ± 0.05 | 2.14 ± 0.93 | 31.52 ± 0.41 |
| 12 | — | — | mPEG-PLA | Tween 80 | 106.3 ± 0.58 | 0.198 ± 0.01 | 3.19 ± 0.29 | 23.80 ± 0.51 |
| 13 | GMS | Oleic acid | — | Tween 80 | 163.3 ± 1.53 | 0.447 ± 0.03 | 1.16 ± 0.84 | 35.48 ± 0.59 |
| 14 | — | Oleic acid | mPEG-PLA | Tween 80 | 102.0 ± 1.00 | 0.139 ± 0.01 | 2.43 ± 0.68 | 48.41 ± 1.70 |

Out of these batches, batch no 1 and 5 was selected further for scale up by High Pressure Homogenization technique. This selection was based on the data of particle size, PDI, shape of particle size distribution graph and % encapsulation efficiency.

### Example 2: Clobetasol Propionate Loaded LPH Nanoparticles (CP LPH NP) by High-Pressure Homogenization Method

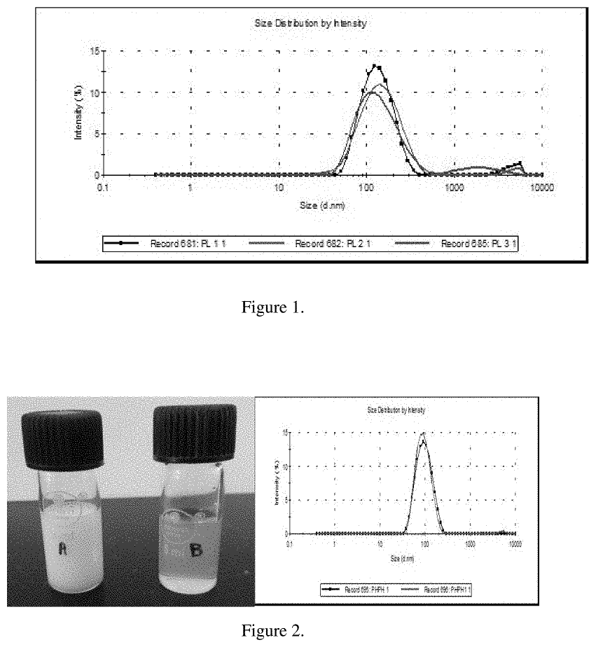

Clobetasol propionate (107 mg), solid lipid (GMS or Precirol® ATO 5; 1600 mg), liquid lipid (oleic acid or linoleic acid; 1600 mg) and polymer (methoxypolyethyleneglycol-co-polylactic acid copolymer (mPEG-PLA); 1600 mg) and were taken in a 100 ml beaker. To this chloroform (7 ml) was added and warmed to 40° C. for obtaining a clear solution. Chloroform was then removed by heating at 70° C. for 3 h to form a uniform matrix. An aqueous solution containing tween 80 (1.5% w/v; 50 ml) was added to the matrix and subjected to high shear homogenization at 30,000 rpm for 5 min to obtain a coarse dispersion. Tween 80 (1.5% w/v; 30 ml) was used to replace the water present in the reservoir and connectors of the high-pressure homogenizer (HPH). The coarse dispersion was then added to the reservoir of HPH and homogenized at a pressure of 1000 bars for 5 min. The temperature during homogenization was monitored and was maintained below 45° C. The nanodispersion (70 ml) was collected at the end of the process, immediately cooled in an ice bath and centrifuged at 5000 rpm for 5 min to remove the unentrapped drug and large particles. The supernatant containing nanoparticles was collected analyzed for particle size and size distribution, and zeta potential using Malvern Zetasizer and drug content in the dispersion using HPLC. Results obtained are shown in table 2. FIG. 2 shows the particle size distribution graph of CP loaded LPH nanoparticles of batch 5 prepared using high pressure homogenizer.

TABLE 2
Characterization of CP loaded LPH nanoparticles
prepared using high pressure homogenizer

| Characterization | Batch 1 | Batch 5 |
|---|---|---|
| Particle size (nm) | 96.5 ± 2.12 | 94.78 ± 7.33 |
| PDI | 0.269 ± 0.003 | 0.213 ± 0.06 |
| Concentration of CP (mg/ml) | 1.03 ± 0.01 | 1.04 ± 0.03 |

### Example 3: Preparation of docetaxel (DTX), cholecalciferol (CVD) or Coenzyme Q10 (CoQ10) loaded LPH nanoparticles

Docetaxel trihydrate, or Coenzyme Q10 (20 mg), solid lipid (Precirol® ATO 5; 180 mg), liquid lipid (linoleic acid; 180 mg) and methoxypolyethyleneglycol-co-polylactic acid copolymer (mPEG-PLA) (180 mg) were taken in a round-bottomed flask. To this chloroform (0.8 ml) was added and warmed to 40° C. for obtaining a clear solution. Chloroform was then removed by heating at 60° C. for 30 min to form a uniform film. An aqueous solution containing tween 80 (1.5% w/v; 10 ml) heated to 60° C. was added to the melted lipid in RBF at 60° C. An aqueous suspension of lipidic material was transferred to 15 ml vial and sonicated at 20% amplitude for 2 min using probe sonicator keeping the temperature at 60° C. The nanodispersion was immediately cooled in an ice bath and centrifuged at 5000 rpm for 5 min to remove the unentrapped DTX and large particles. The supernatant containing nanoparticles was collected analyzed for particle size and size distribution, and zeta potential using Malvern Zetasizer and drug content in the dispersion using HPLC. Process for preparing CVD LPH NP was similar to DTX/CoQ10 but differed with respect to final formula i.e. CVD 7 mg, Precirol® ATO 5 (90 mg), methoxypolyethyleneglycol-co-polylactic acid copolymer (mPEG-PLA) (90 mg) and linoleic acid (90 mg). Results obtained are shown in table 3.

TABLE 3
Characterization of LPH nanoparticles loaded with
docetaxel, cholecalciferol or CoenzymeQ10 (n = 3)

| | Docetaxel | Cholecalciferol | Coenzyme Q10 |
|---|---|---|---|
| Parameter | (DTX) LPH | (CVD) LPH | (CoQ10) LPH |
| Particle size (nm) | 181.37 ± 2.03 | 123.1 ± 6.16 | 121 ± 11.61 |
| PDI | 0.350 ± 0.05 | 0.234 ± 0.03 | 0.252 ± 0.073 |
| % Encapsulation | 78.17 ± 1.90 | 76.80 ± 1.36 | 78.57 ± 3.88 |
| efficiency | | | |
| Zeta potential (mV) | −2.07 ± 0.94 | −4.33 ± 0.85 | −20.23 ± 6.67 |

### Example 4: Preparation of Gel Containing Clobetasol Propionate (CP), Cholecalciferol (CVD) or Coenzyme Q10 (CoQ10) Loaded LPH Nanoparticles

Clobetasol propionate loaded LPH nanoparticles containing gel (˜0.05% w/w of clobetasol propionate; 100 g), cholecalciferol loaded LPH nanoparticles containing gel (0.005% w/w and 0.0025% w/w of CVD, 100 g) or Coenzyme Q10 loaded LPH nanoparticles containing gel (˜0.06% w/w and 0.03% w/w of CoQ10; 100 g) were prepared using 0.75% w/v carbopol 974P. For preparing these three different gels, carbopol 974P (0.75 g) were taken separately in three separate beaker followed by addition of purified water (10 ml) and kept overnight for hydration. Clobetasol propionate loaded LPH nanoparticle dispersion (˜0.05 g and 0.025 g of clobetasol propionate), CVD loaded LPH nanoparticles dispersion (0.005 g and 0.0025 g of CVD) and CoQ10 loaded LPH nanoparticle dispersion (˜0.06 g and 0.03 g of CoQ10 were added to the hydrated carbopol 974P and mixed uniformly using a glass rod.

Propylene glycol (13 g), methylparaben (0.3 g) and propylparaben (0.3 g) were added to the above mixture and stirred vigorously using glass rod until visual homogeneity was obtained. Purified water was added to make up the weight to 95 g and pH of the mixture was adjusted to 6.8 by using 1 M NaOH to obtain a gel. Purified water was then added to make up the weight to 100 g and stirred well to obtain a uniform gel.

### Example 5: In-Vitro Drug Release of Clobetasol Propionate (CP) and Docetaxel (DTX) from LPH Nanoparticles

In-vitro drug release study of clobetasol propionate from freshly prepared gel containing drug loaded LPH nanoparticles and were compared with gel containing drug loaded LPH nanoparticles stored at 2-8° C. and room temperature (RT) for 3 months and 6 months.

Release study for clobetasol propionate (CP) and docetaxel DTX) LPH nanoparticles were carried out in a dialysis bag (SnakeSkin® Dialysis Tubing, ThermoScientific) using media of following composition:

For CP: Sodium lauryl sulfate-10 g, sodium azide-2 g, ethanol-20 ml in phosphate buffer saline (pH 7.4) 1000 ml.

For DTX: Tween 80-10 g, sodium azide-2 g, ethanol-20 ml in phosphate buffer saline (pH 7.4)-1000 ml.

Dialysis bag containing the clobetasol propionate free drug (free CP), a gel containing free clobetasol propionate (CP), gel containing clobetasol propionate loaded LPH nanoparticles (CP LPH NP) stability samples i.e. gel containing drug loaded LPH NP stored at 2-8° C. and room temperature (RT) for 3 months and 6 months respectively, free docetaxel and DTX LPH nanoparticle (DTX LPH NP) were placed in 30 ml of release media and kept in an incubator at 37° C. Samples were taken at following time points 0.25, 0.5, 1, 2, 4, 8, 12, 24, 48, 72, 96, 120, 144 and 168 h.

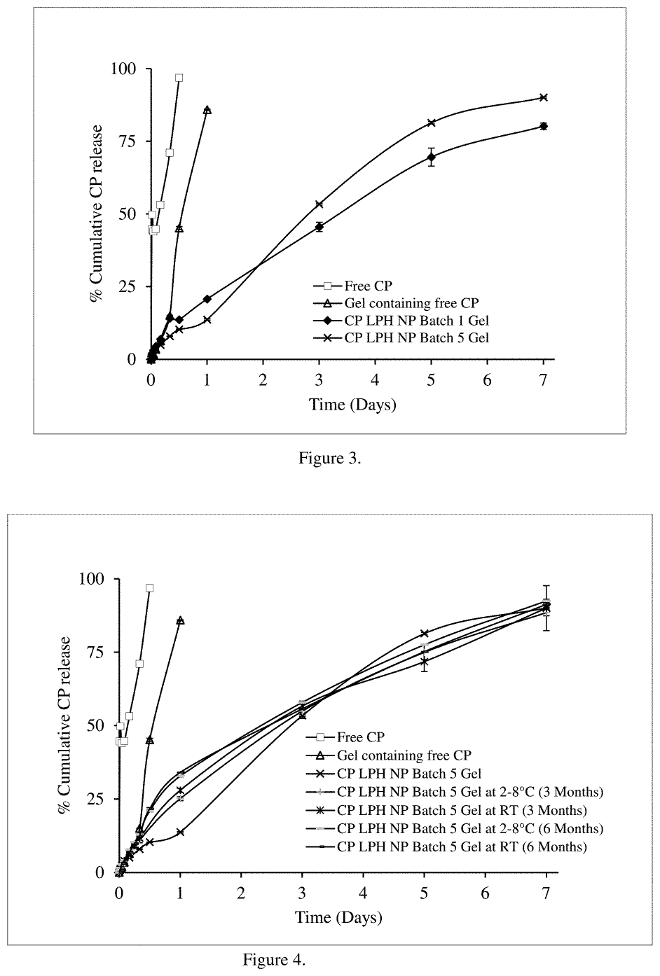

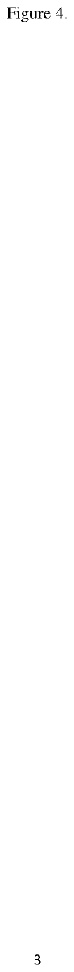

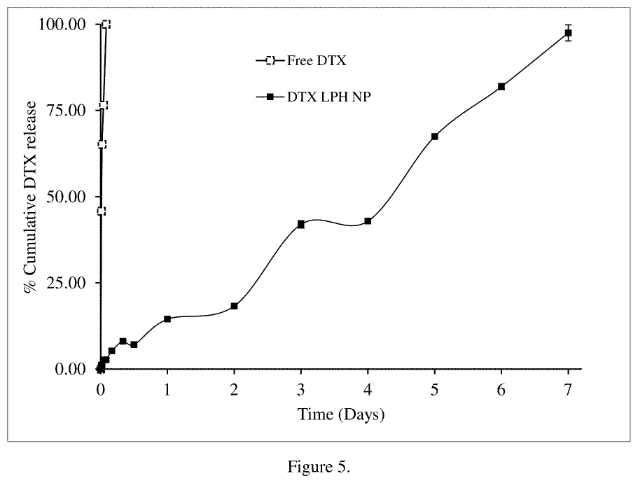

The release profiles of CP from freshly prepared LPH nanoparticles containing gels and stability samples is shown in FIGS. 3 and 4, respectively. Release profile of DTX from LPH nanoparticles is shown in FIG. 5. A sustained release without any burst effect was observed in all the release profiles.

Further, in-vitro drug release profiles of stability samples were found to be similar to that of freshly prepared gel containing CP LPH nanoparticles with no burst release.

### Example 6: In-Vitro Cytotoxicity Assay of Clobetasol Propionate (CP), Docetaxel (DTX), Cholecalciferol (CVD) Loaded LPH Nanoparticles

For clobetasol propionate (CP) and cholecalciferol (CVD), in-vitro cytotoxicity assay was performed using MTT assay in HaCaT cells whereas in-vitro cytotoxicity assay for docetaxel, was performed using MTT assay in 4T1 breast cancer cells (murine mammary carcinoma cell line). Briefly, 5×10³ cells/well were seeded in 96 well culture plates and allowed to adhere for 24 h (incubated at 37° C., 5% CO₂). For clobetasol propionate, the cells were treated with free drug (free CP) or drug loaded LPH nanoparticles (CP LPH NP) within the range of 1-100 μM for 48 h. For CVD, the cells were treated with free drug (free CVD) or drug loaded LPH nanoparticles (CVD LPH NP) within the range of 1-500 nM for 48 h. For DTX, cells were treated with free drug (free DTX) or drug loaded LPH nanoparticles (DTX LPH NP) within the range of 1-1000 nM for 48 h.

The DMSO and blank nanoparticles were used as a control. After 48 h, the culture media was removed and plates were washed with sterile PBS (pH 7.4). The cells were treated with 20 μL of MTT reagent (5 mg/mL) added to each well with 100 μL of serum free culture media for 4 h in dark condition. After 4 h, the media were removed from the plate and formazan crystals were dissolved in molecular grade DMSO. The absorbance was recorded at 560 nm and 630 nm as reference wavelength. The percentage cell inhibition was determined by comparison with untreated cells.

% Cell inhibition=(OD sample wells/OD control wells)×100

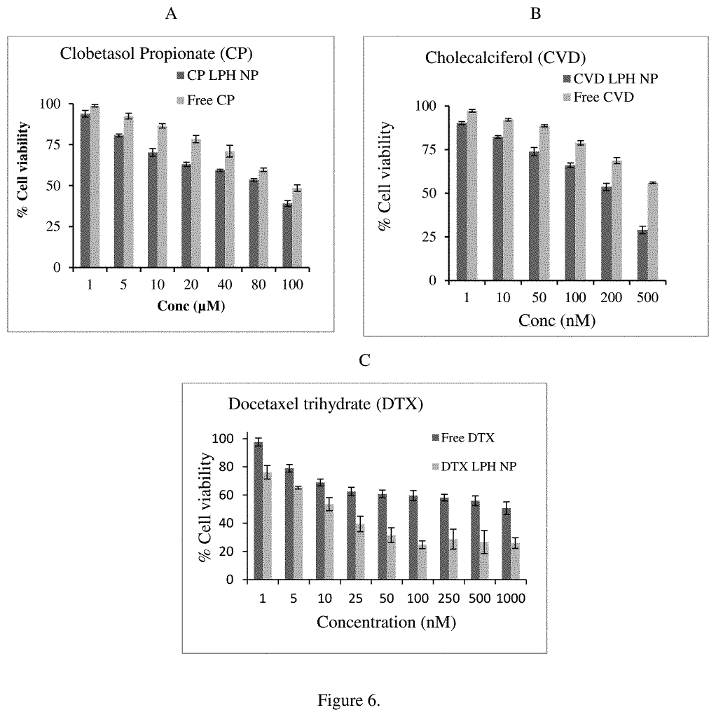

As shown in FIGS. 6A and B, the growth of HaCaT cells were significantly reduced upon incubation with clobetasol propionate (CP) or cholecalciferol (CVD) loaded LPH nanoparticles in comparison to free drugs. In a similar fashion significantly higher cytotoxicities against 4T1 breast cancer cells were observed for docetaxel loaded LPH nanoparticles (DTX LPH NP) as compared to free DTX (FIG. 6C).

### Example 7: In-Vivo Efficacy Evaluation of Gel Containing Clobetasol Propionate (CP), Cholecalciferol (CVD) or Coenzyme Q10 (CoQ10) Loaded LPH Nanoparticles in Imiquimod Induced Psoriasis Model in Swiss Albino Mice

Swiss albino mice (8 to 12 weeks) were subjected with topical dose of 62.5 mg of commercially available IMQ cream (5%) that was applied on the shaved back and right ear for 5 consecutive days, translating in a daily dose of 3.125 mg of the active compound. Animals were divided into following groups: negative control, positive control, marketed CP gel (Clobetamos® which contains CP equivalent to 0.05% w/w), plain gel containing CVD (0.005% w/w), CoQ10 (0.06%) and gel containing LPH nanoparticles (LPH NP) loaded with respective drugs, clobetasol propionate (CP), cholecalciferol (CVD) or coenzyme Q10 at similar dose as that of plain gels (i.e 0.05% w/w for CP, 0.005% w/w for CVD, 0.06% w/w for CoQ10) and at half doses as that of plain gels (i.e 0.025% w/w for CP, 0.0025% for CVD and 0.03% w/w for CoQ10). Negative control mice were left without any treatment. Positive control mice were subjected to IMQ treatment only so as to induce psoriatic like skin condition. Animals were treated with products weighing 40 mg/cm² once daily. The efficacy of the treatment was determined by an objective scoring system that was developed based on the clinical Psoriasis Area and Severity Index (PASI). Cumulative scoring (erythema plus scaling plus thickening) was done on a scale from 0 to 12 indicated the extent of inflammation. Ear thickness (both right and left) were measured using micrometer. Here left ear served as a control. The back skin thickness was measured using Vernier caliper. Increase in the thickness of right ear and back skin indicated the extent of psoriasis. At the end of study animals were sacrificed and their back skin and right ear, were examined histologically.

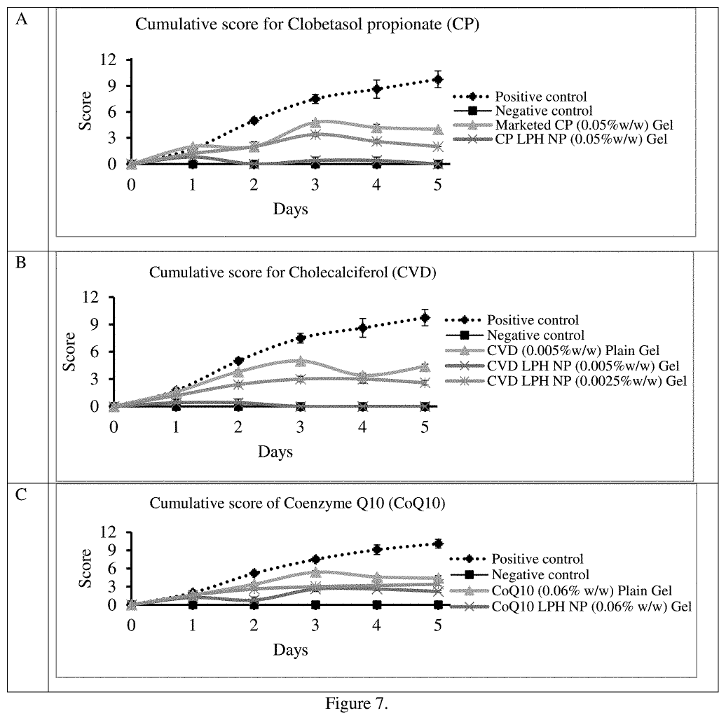

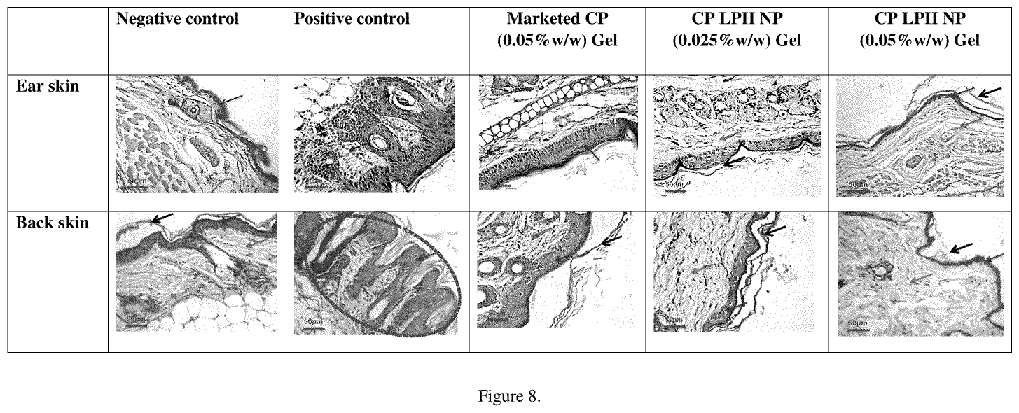

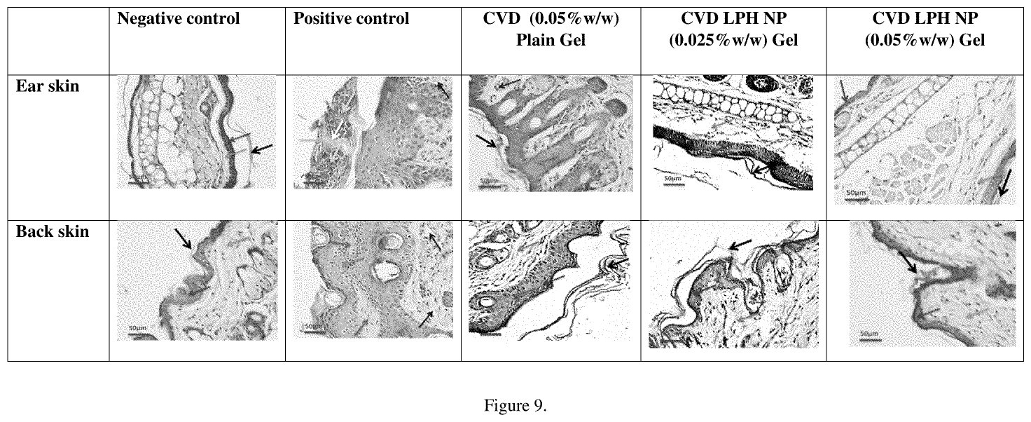

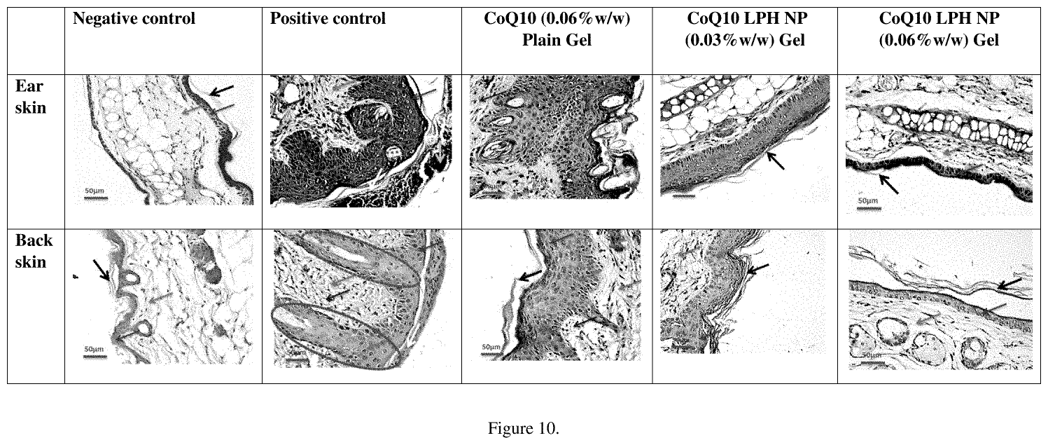

As shown in FIG. 7, in positive control cumulative score started increasing significantly from day 1 and got progressively increased till day 5. From the data it can be observed that both marketed CP gel, plain gels of CVD, CoQ10 and test group containing LPH nanoparticles loaded gel containing clobetasol propionate (CP), cholecalciferol (CVD) or coenzyme Q10 loaded LPH nanoparticles at full dose and half dose are able to treat psoriasis. Test groups that were treated with drug loaded LPH nanoparticle containing gel showed improved efficacy as compared to marketed formulation or plain gels (i.e free drugs without nanoparticles) (FIG. 5A to 5C). Further histopathological studies (both right ear and back skin) showed marked improvement in the efficacy of clobetasol propionate (CP), cholecalciferol (CVD) or coenzyme Q10 by incorporating in the LPH nanoparticle system (FIGS. 8, 9 and 10).

### Example 8: Ex-Vivo Study to Determine the Quantity of Drug in Deeper Skin Layers (Viable Epidermis and Dermis)

The ex-vivo skin permeation study was performed on the psoriatic skin of Swiss albino mice using Franz diffusion cells with the contact surface area of 1 cm². Shaved mice's skin was mounted between the donor and receptor compartment that was held tightly by clamps. The receptor compartment was filled with PBS (5 ml; pH 7.4) containing sodium lauryl sulphate (1% w/v) and ethanol (2% v/v).

In the donor compartment, marketed clobetasol propionate (CP) gel (Clobetamos®) or clobetasol propionate loaded LPH nanoparticle gel (equivalent to 25 μg of clobetasol propionate) was taken followed by incubation for 24 h at 37±1° C. with a stirring speed of 800 rpm. After 24 h, aliquots were withdrawn from the receptor compartment and skin samples were unclipped from the Franz diffusion cells, washed three times with PBS (pH 7.4) followed by air drying. A 19 mm Scotch (3M, USA) cellophane tape was used for tape stripping. The first stripped tape was discarded as it contains the unabsorbed drug. For the removal of stratum corneum (SC) layer, 15 strips were detached in such a way that the whole area of the tape was utilized. After removing stratum corneum from skin samples, remaining skin was soaked in methanol and sonicated for 1 h for complete extraction of the drug from deeper layers of skin i.e. viable epidermis and dermis. The samples were analyzed by the developed bioanalytical RP-HPLC method. The analysis was performed on Shimadzu HPLC system equipped with a PDA detector. Chromatographic separation was performed on Inertsil-C18 ODS column (5 μm, 4.6×250 mm) with a mobile phase consisting of acetonitrile: water (60:40) run at a flow rate of 1 mL/min. Docetaxel (DTX) was used as an internal standard. The injection volume was 60 μL and the retention time for CP and DTX was found to be 13.29 min and 7.74 min, respectively.

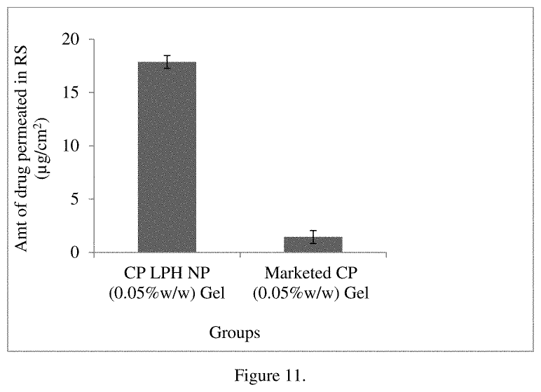

The results are shown in FIG. 11. This study gives information regarding the amount of the drug that permeated to deeper dermal layers (RS) i.e. viable epidermis and dermis. From the FIG. 11, it can be observed that, in case of marketed formulation, most of the drug did not penetrate deeper layers of the skin, whereas in the case of nanoparticle loaded gel there was significantly higher penetration of drug (71.47% of drug per cm²) in deeper layers as compared to marketed CP gel (5.8% of drug per cm 2).

### Example 9: To Determine the Pharmacokinetics (Systemic Absorption) of Clobetasol Propionate from Gel Containing Clobetasol Loaded LPH Nanoparticle Upon Topical Application on IMQ Induced Psoriatic Mice

IMQ induced psoriatic Swiss albino mice (25-30 g) were divided into two groups (n=6); marketed formulation treated groups and test groups. Marketed formulation treated groups group animals were treated with Clobetamos® (Clobetasol propionate equivalent to 0.05% w/w) and test group animals were treated with clobetasol propionate loaded LPH nanoparticle (CP LPH NP) containing gel (Clobetasol propionate equivalent to 0.05% w/w). At specific time points (i.e. 0.5 h, 1 h, 3 h, 6 h, 12 h and 24 h) blood (100 μl) from animals were withdrawn and pooled that was further centrifuged at 6500 RPM for 15 min to isolate plasma. Plasma samples were analyzed by reverse-phase HPLC using the developed bio-analytical method to determine the amount of drug that has reached systemic circulation. The analysis was performed on Shimadzu HPLC system equipped with a PDA detector. Chromatographic separation was performed on Inertsil-C18 ODS column (5 μm, 4.6×250 mm) with a mobile phase consisting of acetonitrile: water (55:45) run at a flow rate of 1 mL/min. Docetaxel (DTX) was used as an internal standard. The injection volume was 60 μL and the retention time for CP and DTX was found to be 20.6 min and 10.4 min, respectively.

The results showed that in the case of the marketed group treated with Clobetamos®, there was the presence of quantifiable levels of drug detected in plasma from 30 min to 6 h after topical application. In the case of a test group treated with gel containing clobetasol loaded LPH nanoparticle (CP LPH NP), there were no quantifiable levels of drug detected in plasma over a period of four days (Table 4). As the CP LPH nanoparticles released the drug slowly in a sustained manner along with specific skin retentive capability with no systemic delivery, it can be concluded that the systemic side effects associated with the drug get reduced which is not in the case with Clobetamos®.

TABLE 4
Systemic absorption of clobetasol propionate in IMQ
induced psoriatic mice model

| | | | | | | Gel | CP | LPH |
|---|---|---|---|---|---|---|---|---|
| | | | | | | nanoparticles | | |
| | Clobetamos ® | | | Average | | containing gel | | |
| Time | (ng/ml) | | | concentration | | (ng/ml) | | |
| (h) | 1 | 2 | 3 | SEM | | 1 | 2 | 3 |
| 0.5 | 20.02 | 56.85 | 3.95 | 26.94 | 11.07 | ** | ** | ** |
| 1 | 66.72 | 102.42 | 112.31 | 93.81 | 9.79 | ** | ** | ** |
| 3 | ND | 77.03 | 102.07 | 89.55 | 7.23 | ** | ** | ** |
| 6 | 55.33 | 30.22 | 35.84 | 40.47 | 5.38 | ** | ** | ** |
| 12 | * | ** | * | * | | ND | ** | ** |

Abbreviation: ND: Not determined, *: Detectable but not quantifiable, **: Not detectable and not quantifiable

We claim:

1. A lipid-polymer hybrid nanoparticles comprises a solid lipid, a liquid lipid and an amphiphilic polymer.

2. The lipid-polymer hybrid nanoparticles according to claim 1 comprises a solid lipid, a liquid lipid and an amphiphilic polymer, wherein (a) core comprises of a solid lipid, a liquid lipid and hydrophobic block of polymer and (b) shell surrounding the core comprises of a hydrophilic portion of polymer.

3. The lipid-polymer hybrid nanoparticles according to claim 2 wherein entrap one or more active agents within a hydrophobic core formed due to interaction of a solid lipid, a liquid lipid and hydrophobic block of an amphiphilic polymer.

4. The lipid-polymer hybrid nanoparticles according to claim 2 wherein a ratio of a solid lipid to a liquid lipid is in range of about 10:1 to about 1:10

5. The lipid-polymer hybrid nanoparticles according to claim 2 wherein a ratio of a solid lipid to an amphiphilic polymer is in range of about 10:1 to about 1:10.

6. The lipid-polymer hybrid nanoparticles according to claim 2 wherein a ratio of a liquid lipid to an amphiphilic polymer is in range of about 10:1 to about 1:10.

7. The lipid-polymer hybrid nanoparticles according to claim 1 or claim 2 further comprises at least one surfactant.

8. The lipid-polymer hybrid nanoparticles according to claim 7 comprises
(a) a core comprises of a solid lipid, a liquid lipid and hydrophobic block of polymer;
(b) an active molecule entrapped in core;
(c) shell surrounding core comprises of hydrophilic portion of polymer and
(d) a surfactant.

9. The lipid-polymer hybrid nanoparticles according to claim 8 wherein a surfactant present in the aqueous phase surrounding the nanoparticles.
